(19) United States
(12) Patent Application Publication
(10) Pub. No.: US 2013/0144263 A1
(43) Pub. Date: Jun. 6, 2013

(54) BALLOON CATHETER SYSTEM

(71) Applicants: Eyal Teichman, Hod-HaSharon (IL); Guy Kotlizky, Kfar-Shemaryahu (IL)

(72) Inventors: Eyal Teichman, Hod-HaSharon (IL); Guy Kotlizky, Kfar-Shemaryahu (IL)

(21) Appl. No.: 13/691,799

(22) Filed: Dec. 2, 2012

Related U.S. Application Data
(60) Provisional application No. 61/629,959, filed on Dec. 2, 2011.
(60) Provisional application No. 61/649,318, filed on May 20, 2012.

(51) Int. Cl. A61M 25/10

(52) U.S. Cl. CPC A61M 25/104; A61M 25/1011
USPC 604/509; 606/192; 604/101.02

(57) ABSTRACT

A balloon catheter system is provided. The catheter includes a first balloon disposed around a second balloon which is movable within the first balloon.

### RELATED APPLICATIONS

This application claims the benefit of priority under 35 USC §119(e) from U.S. Provisional Patent Application Nos. 61/629,959 filed on Dec. 2, 2011 and 61/649,318 filed on May 20, 2012. The contents of the above applications are all incorporated by reference as if fully set forth herein in their entirety.

### FIELD AND BACKGROUND OF THE INVENTION

The present invention relates to a balloon catheter system and to a method of using same for opening narrowed biological vessels such as stenosed arteries. The present invention also relates to a system for delivering a composition to the wall of a treated biological vessel.

Peripheral vascular disease (PVD) is a common condition with variable morbidity affecting mostly men and women older than 50 years. Intermittent claudication (pain while walking that abates during rest) is the most common symptom of PVD; other symptoms include numbness or weakness in the legs, aching pain in the feet or toes while at rest, non-healing ulcers on the leg or foot, cold legs or feet, and skin color changes of the legs or feet.

Some patients diagnosed with the disease remain stable or improve with conservative management, those who do not, undergo surgical or percutaneous intervention. Surgical bypass is the gold standard for extensive vascular occlusive disease, but endovascular intervention such as percutaneous transluminal angioplasty is being used more frequently, particularly in patients with significant comorbid conditions.

Percutaneous transluminal angioplasty (PTA) is a procedure in which a thin, flexible tube called a catheter is inserted through an artery and guided to the place where the artery is narrowed. When the tube reaches the narrowed artery, a small balloon at the end of the tube is inflated for 20 seconds to 3 minutes. The pressure from the inflated balloon forces the plaque material (typically fat and calcium) against the wall of the artery to open the vessel and improve blood flow.

Although angioplasty can be quite effective in opening blockages in coronary arteries where the blockage is relatively short, in peripheral arteries where blockages can span several centimeters, balloon angioplasty can result in focal stenosis, a condition in which a short region of the lesion remains unopened following ballooning.

While reducing the present invention to practice, the present inventors have devised a system which can be used for angioplasty of narrowed vessels as well as treat focal stenosis resulting from such angioplasty.

### SUMMARY OF THE INVENTION

According to one aspect of the present invention there is provided an angioplasty system comprising a catheter being mountable on a guide-wire, the catheter including a first balloon disposed around a second balloon, the second balloon being movable within the first balloon.

According to further features in preferred embodiments of the invention described below, the second balloon is movable along a longitudinal axis of the first balloon.

According to still further features in the described preferred embodiments the first balloon is disposed co-axially around the second balloon.

According to still further features in the described preferred embodiments the first balloon is a non/semi-complaint balloon.

According to still further features in the described preferred embodiments the second balloon is a non-compliant balloon.

According to still further features in the described preferred embodiments the first balloon is inflatable to a pressure of up to about 12 atm.

According to still further features in the described preferred embodiments the second balloon is inflatable to a pressure of up to about 30 atm.

According to still further features in the described preferred embodiments the first balloon is 80-300 mm preferably 80-200 mm long when fully inflated.

According to still further features in the described preferred embodiments the second balloon is 8-50 mm long when fully inflated.

According to still further features in the described preferred embodiments a conduit for inflating the second balloon is positioned within the catheter side-by-side to a conduit of the guide-wire.

According to still further features in the described preferred embodiments the second balloon is movable within the first balloon by pulling or pushing the conduit with respect to the catheter.

According to still further features in the described preferred embodiments the first and the second balloons are separately inflatable.

According to still further features in the described preferred embodiments the catheter includes 2 tubes, a first tube attached to the first balloon and a second tube attached to the second balloon.

According to still further features in the described preferred embodiments the first tube includes a first lumen for inflating the first balloon and the second tube includes a second lumen for inflating the second balloon.

According to still further features in the described preferred embodiments the first tube and the second balloon can be co-axial or side by side.

According to still further features in the described preferred embodiments an external surface of the first balloon includes cavities.

According to still further features in the described preferred embodiments the cavities are dimples.

According to still further features in the described preferred embodiments the cavities are filled with a drug.

According to still further features in the described preferred embodiments the drug is paclitaxel or sirolimus.

According to another aspect of the present invention there is provided a method of opening a stenosed vessel comprising: (a) inflating a first balloon within a stenosis in the vessel to thereby partially open the stenosis; (b) positioning a second balloon disposed within the first inflatable balloon at a site of residual stenosis; and (c) inflating the second balloon thereby opening the residual stenosis and opening the stenosed vessel.

According to still further features in the described preferred embodiments step (b) is effected while the first balloon is inflated.

According to still further features in the described preferred embodiments the second balloon is movable along a longitudinal axis of the first balloon.

According to still further features in the described preferred embodiments the first balloon is disposed co-axially around the second balloon.

According to still further features in the described preferred embodiments the first balloon is coated with a drug and (a) releases a first dose of the drug.

According to still further features in the described preferred embodiments (c) releases a second dose of the drug from the first balloon.

According to still further features in the described preferred embodiments the method further comprises delivering the first and the second balloon to the stenosis, wherein the second balloon is positioned within a proximal portion of the first balloon during the delivering.

The present invention successfully addresses the shortcomings of the presently known configurations by providing a balloon catheter system that can be used to treat narrowed vessels and any residual focal stenosis resulting from such treatment with a single catheter insertion while also providing a unique and efficient approach for localized drug delivery.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a perspective cut-away view of one embodiment of the system of the present invention.

FIGS. 2A-B are cross sectional drawings illustrating the various components of the system of FIG. 1; FIG. 2b provides magnified views of the regions circled in FIG. 2a.

FIGS. 3A-B illustrate a cross sectional view of another embodiment of the system of the present invention. FIG. 3b is a magnified view of a proximal portion of the system shown in FIG. 3a.

FIGS. 4-7 illustrate the steps of positioning (FIG. 4) and inflating (FIG. 5) the external balloon, and positioning (FIG. 6) and inflating (FIG. 7) the internal balloon in a narrowed vessel.

FIGS. 8A-D are isometric views of an embodiment of the present system showing the complete system (FIG. 8a), the balloon end of the catheter (FIG. 8b) and cutaway views of the balloon end (FIG. 8c) and handle (FIG. 8d).

FIGS. 9A-B illustrate a rapid exchange embodiment of the present system shown in isometric (FIG. 9a) and cutaway (FIG. 9b) views.

FIG. 10 illustrates a drug delivery configuration of the present system.

FIG. 11 illustrates the connector (handle) end of the prototype of the present system along with a plunger pump used for inflating the balloons of the prototype.

FIGS. 12A-E illustrate bench testing of the prototype system of FIG. 11 through inflation/deflation cycles and movement of the internal balloon within the external balloon.

### DESCRIPTION OF THE PREFERRED EMBODIMENTS

The present invention is of a system which can be used for angioplasty. Specifically, the present invention can be used to open narrowed vessels and particularly blood vessels such as peripheral arteries, where standard angioplasty can be ineffective due to formation of focal stenosis following ballooning.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Focal stenosis is one of the complications of angioplasty in peripheral blood vessels. Several approaches for dealing with focal stenosis have been devised including slow low-pressure inflation of the long balloon, over-inflation of the long balloon or post angioplasty stenting.

These approaches complicate the procedure and can expose the patient to additional radiation.

To traverse such limitations, the present inventors have devised an angioplasty system that utilizes an external balloon (also referred to herein as a first balloon) for opening a narrowed vessel and an internal balloon (also referred to herein as a second balloon) for opening residual stenosis (e.g. focal stenosis) resulting from use of the external balloon. As is further described herein, the internal balloon is positioned within the external balloon and is movable along a longitudinal axis thereof. This enables positioning of the internal balloon at a site of focal stenosis while the external balloon is in position and inflated, and as such, it enables rapid and accurate treatment of focal stenosis. As is further described herein, the angioplasty system of the present invention is also particularly suitable for selective delivery of a drug to specific regions of a vessel.

Thus, according to one aspect of the present invention there is provided an angioplasty system. As used herein, the term angioplasty system refers to a balloon catheter which can be used to force open narrowed vessels such peripheral blood vessels coronary blood vessels, as well as other conduits and ducts such as ureters and the like.

The system of the present invention includes a catheter which is mountable on a guide-wire (e.g. a 0.014-0.035 inch guide-wire) in an over-the-wire configuration or a rapid exchange configuration (also known as a monorail configuration). The catheter includes a first balloon which is disposed around a second balloon, preferably in a coaxial (concentric) configuration.

The first balloon can be fabricated from a non- or semi-compliant material such as PET, Nylons, PE, Polyurethanes, PVC using blow molding, dip molding or extrusion techniques. The first balloon is preferably cigar or torpedo-shaped with a length of 80-300 mm (e.g. 80, 100, 150, 200, 250 or 300 mm long), and a diameter of 3-10 mm. The first balloon can be inflated with air, gas or a liquid (e.g. saline) to a pressure of 2-12 atm.

The second balloon is fabricated from a semi- or preferably non-complaint material such as PET, Nylons, PE, Polyurethanes, PVC using blow molding, dip molding or extrusion techniques. The second balloon is preferably spherical or slightly oblong in shape with a diameter of 3-10 mm and length which is preferably less than half of the first balloon, typically 5-50 mm (e.g. 5, 10, 20, 30, 40 or 50 mm long). The second balloon can be inflated with air, gas or a liquid (e.g. saline) to a pressure of 4-24 atm.

In most cases, the first and second balloons of the present system are of equal diameter. However, in some cases, its advantageous to have a larger diameter second (internal) balloon, e.g. 0.5-2 mm, preferably 1 mm larger in diameter than the first (external) balloon; for example, the present system can include a 5 mm diameter first (external) balloon and a 6 mm diameter second (internal) balloon. A system including a larger diameter second balloon can be used in cases where the stenosis includes angioplasty-resistant calcified regions.

The catheter and balloons are configured such that the second balloon is movable within the first balloon along a longitudinal axis thereof. By enabling movement of the second balloon within the first balloon, the present system enables use of the second balloon in opening any residual focal stenosis while the first balloon is inflated and positioned across the plaque, thus negating the need for deployment of an additional system (e.g. a second angioplasty system).

The first and second balloons are separately attached to the catheter in order to enable separate inflation and movement. Several configurations for enabling such functionality can be used with the present system.

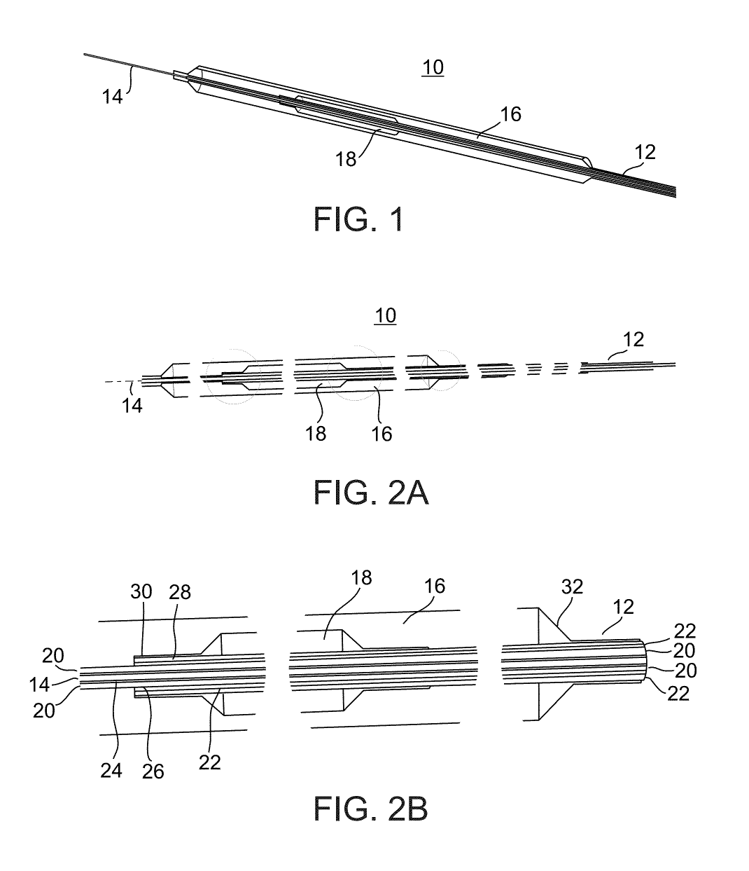

FIGS. 1-2b illustrate one embodiment of the present system which is referred to herein as system 10.

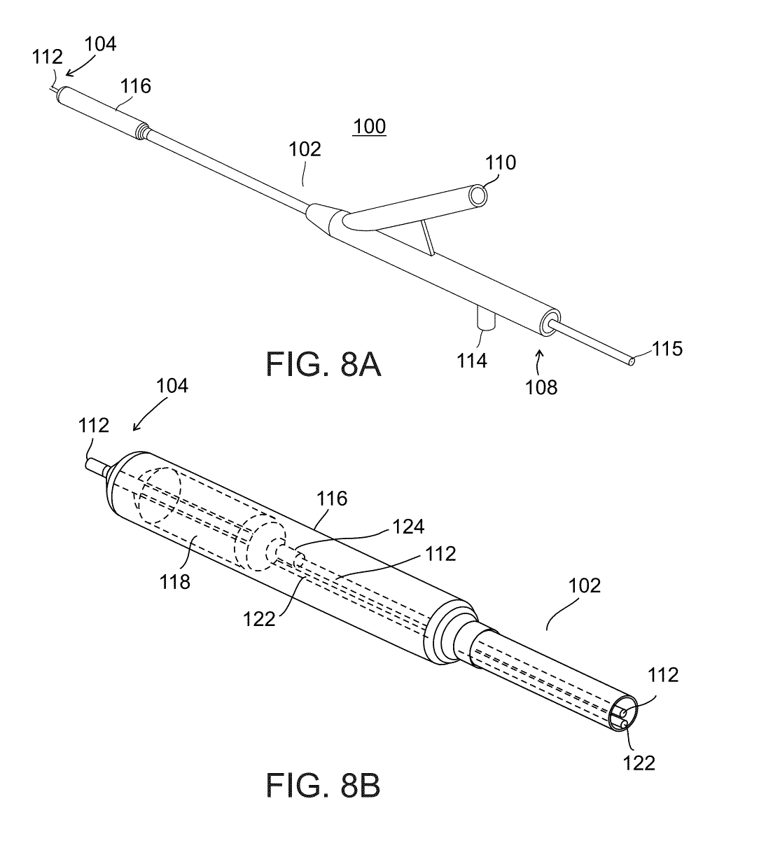

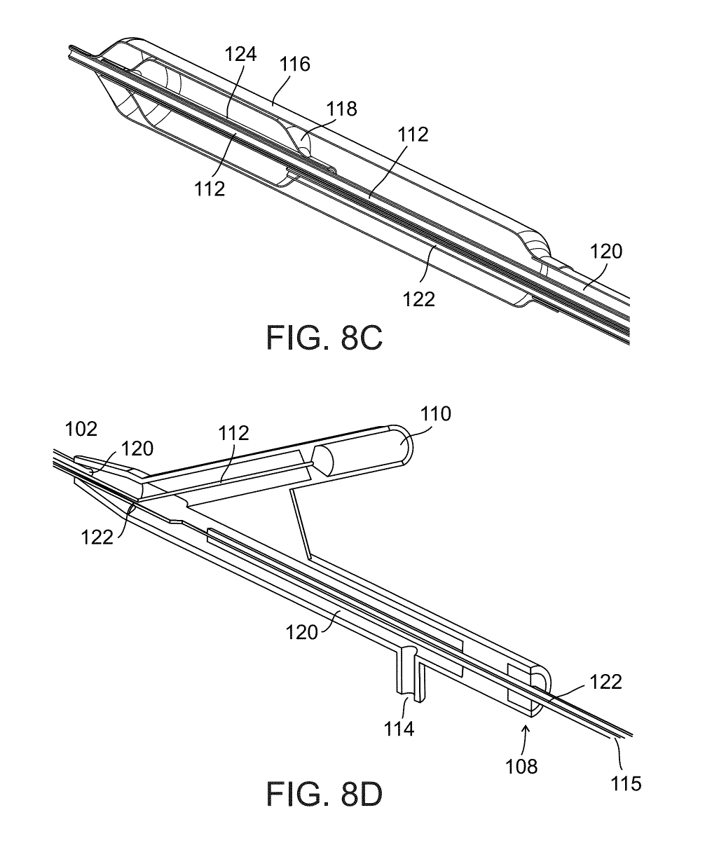

System 10 includes a catheter 12 which is configured for over-the-wire operation. In that respect, catheter 12 has an inner lumen which is 0.36-0.9 mm in diameter and thus can accommodate a guide-wire 14. Catheter 12 is preferably 100-150 cm in length with an external diameter of 4-8 Fr (1.3-2.6 mm). Catheter 12 includes a user operable connector/handle which is described in greater detail in FIG. 8a-d.

Catheter 12 includes an external balloon 16 and an internal balloon 18 which, in this embodiment of system 10, is concentric with external balloon 16 (defined by outer wall 32). External balloon 16 is in fluid communication with conduit 20, while internal balloon 18 is in fluid communication with a conduit 22. Conduits 20 and 22 can be used to separately inflate balloon 16 and 18 using a fluid source positioned outside the body and in communication with conduit ports positioned at a proximal end of catheter 12.

As shown in FIG. 2b, conduit 20 is formed between tubes 24 and 26, and conduit 22 is formed between tubes 26 and 28. Balloon 18 is moved along the longitudinal axis of catheter 12 (within balloon 16) by pushing/pulling tubes 28 and 26 relative to tube 24. Tube 24 is mounted over guidewire 14 and is advanced thereupon to position system 10 in a lumen of a blood vessel.

The tubes of system 10 can be fabricated from Nylon, Pebax, HDPE, LDPE, PTFE, Polyimide and may be braid re-enforced. The walls of tubes can be 12.5 μm-0.4 mm thick, and a gap between tubes (to form conduit) can be 25 μm-0.15 mm.

System 10 is assembled by first gluing tube 24 to the distal end of balloon 16. Tubes 26 and 28 are then glued to the distal end of balloon 18 and tube 28 is also glued to the proximal end of balloon 18. Holes are made in tube 28 to establish a fluid connection with balloon 18. Balloon 18 is then threaded over tube 24 and into Balloon 16. Finally, tube 12 is glued to the proximal end of balloon 16.

In such a configuration of system 10, tubes 26 and 28 are glued at their distal ends, and as such no sealing is required distally to balloon 18. In addition, tube 24 is glued to distal end of balloon 16 and as such, no sealing is required distally to balloon 16. Sealing is required in two places, between tubes 24 and 26 and between tubes 12 and 28 (at the proximal end).

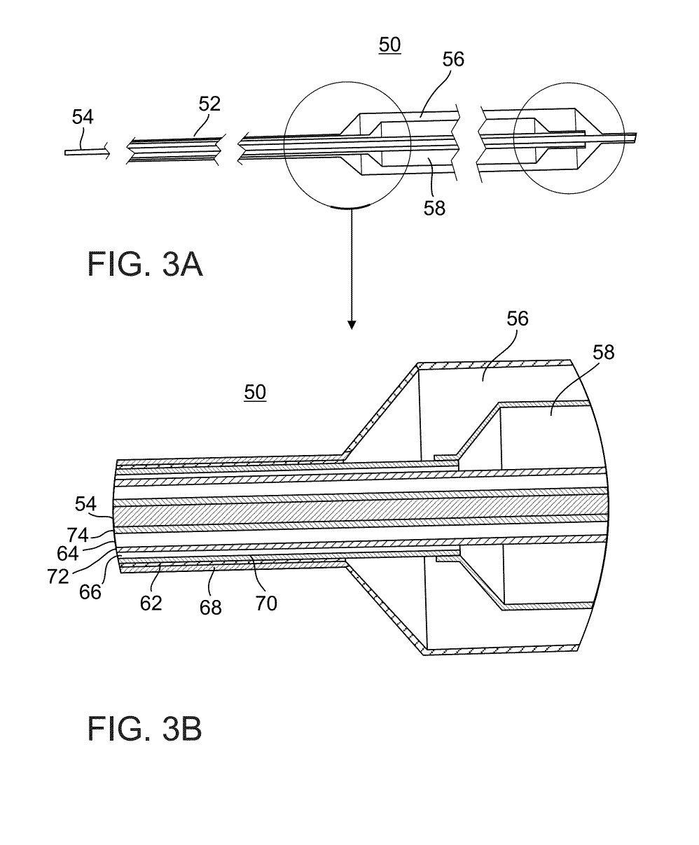

FIGS. 3a-b illustrate another configuration of the present system which is referred to herein as system 50.

System 50 includes a catheter 52 which is configured for over-the-wire operation. In that respect, catheter 52 has an inner lumen 15 which is 0.36-0.9 mm in diameter and thus can accommodate a guide-wire 54. Catheter 52 is preferably 100-150 cm in length with an external diameter of 4-8 Fr (1.3-2.6 mm).

Catheter 52 includes an external balloon 56 and an internal balloon 58 which, in this embodiment of system 50, is concentric with external balloon 56. External balloon 56 is in fluid communication with conduit 62 or 64. Internal balloon 58 is in fluid communication with a conduit 66 which lies between conduit 62 and conduit 64.

Conduits 62 or 64 can be used to inflate balloon 56, while conduit 66 can be used to inflate balloon 58 using a fluid source positioned outside the body and in communication with conduit ports positioned at a proximal end of catheter 52.

Conduit 62 is formed between tubes 68 (discontinuous) and 70 (continuous) while conduit 64 is formed between tubes 72 and 74.

Conduit 66 which is used to fill internal balloon 58 is formed between tubes 70 and 72.

Tube 74 is mounted over guidewire 54 and is advanced thereupon to position system 50 in a lumen of a blood vessel.

Internal balloon 58 is moved within balloon 60 by sliding tubes 70 and 72 relative to tubes 68 and 74.

The tubes of system 50 can be fabricated from Nylon, Pebax, HDPE, LDPE, PTFE, Polyimide and may be braid reinforced. The walls of tubes can be 12.5 μm-0.4 mm thick, and a gap between tubes (to form conduit) can be 25 μm-0.15 mm.

Catheter 52 can include markers distal and proximal to balloons 56 and 58. For example, markers can be placed over tube 68 (proximal side) and tube 74 (distal side) for balloon 56, and over tube 70 (proximal side) and tube 72 (distal side) for balloon 58. Catheter 52 can also include inflation ports for separately inflating balloons 56 and 58.

System 50 can be fabricated by gluing tube 74 to the distal end of balloon 56, and tube 72 to the distal end of balloon 58. Tube 70 can then be threaded over tube 72 and glued to the proximal end of balloon 58 to create a balloon 58 assembly. This assembly is now threaded over tube 74 into the proximal end of balloon 56. Tube 68 can then be threaded over tube 70 and glued to the proximal end of balloon 56.

The conduit and tube arrangement of system 50 provides several advantages:

(i) sealing is required only in one conduit either 62 or 64 (depending on which is used for inflating balloon 56); and

(ii) using fewer tubes decreases the overall external diameter of system 50 and lowers production costs.

In the embodiments shown in FIGS. 1-3b, the present system is configured for angioplasty of narrowed peripheral arteries. As such, typical working pressure for the external balloon can be in the range of 4-12 atm, while the typical pressure for the internal balloon can be 10-24 atm.

It will be appreciated that since the internal balloon is inflated while positioned within an inflated external balloon (external at 4-12 atm); the pressure rise in the internal balloon can lead to a rise in pressure in the external balloon. To compensate for such a rise in pressure, the external balloon can include a valve for releasing (preferably automatically—e.g. blow off valve) the pressure from the external balloon after the internal balloon is positioned and at least partially inflated.

Since inflation of the internal balloon increases the pressure within the external balloon, the external balloon can include a blow off valve for releasing fluid from the external balloon over a predetermined pressure (e.g. the maximum pressure rating of the balloon).

As is mentioned hereinabove, the internal balloon is configured for opening any focal or residual stenosis which results from use of the external balloon. In order to maneuver the catheter to a plaque region and move the internal balloon within the external balloon, the present system also includes a user actuated mechanism which is mountable on a proximal (extracorporeal) end of the catheter. Such a mechanism (not shown) can provide a user with control over positioning of the external balloon as well as positioning of the internal balloon within the external balloon.

The external and internal balloons can be inflated using approaches well known in the art including syringes pumps and the like.

Preferably, balloon inflation is effected using a single pump and a selector mechanism that enables selective filling of the external and internal balloons. The selector mechanism can have several manually selected modes, a first in which the external balloon is fluidly connected to the pump and the internal balloon is sealed and a second where the internal balloon is fluidly connected to the pump and the external balloon is sealed (or deflated). A third mode can be used to depressurize both balloons.

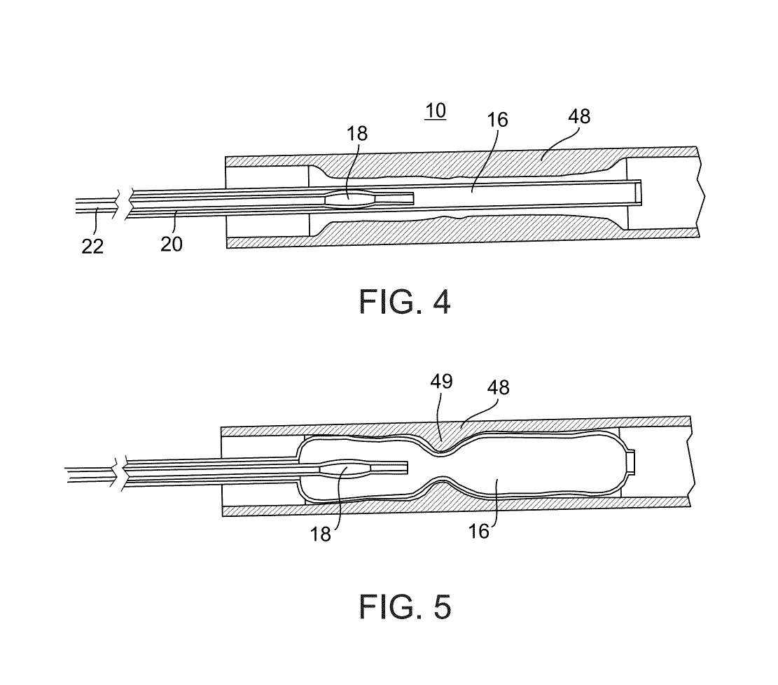

FIGS. 4-7 illustrate use of the present system in opening a narrowed vessel. Although system 10 is illustrated in FIGS. 4-7, alternative system configurations (e.g. system 50 above or system 100 below) can also be used to treat focal stenosis using the steps shown in FIGS. 4-7.

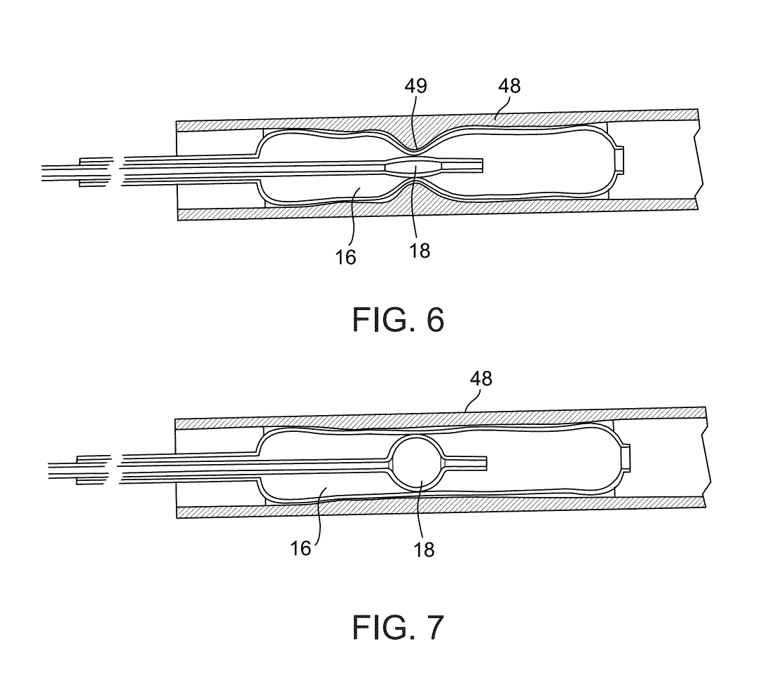

System 10 can be guided to the region of a plaque 48 (FIG. 4) over a guide-wire (not shown) using well known PTA approaches. Once in position, external balloon 16 can be inflated to force open plaque 48 (FIG. 5). Residual stenosis 49 can be located and visualized by introducing a contrast solution into external balloon 16 via conduit 22. Once a focal stenosis 52 is located, internal balloon 18 is moved within (inflated) external balloon 16 and positioned within focal stenosis 49 (FIG. 6). Balloon 18 is then inflated to open focal/residual stenosis 52 (FIG. 7) via conduit 20 and balloon 16 is optionally deflated to compensate for an increase in internal pressure due to inflation of balloon 18. Balloon 18 and 16 are then fully deflated and system 10 is removed from the body.

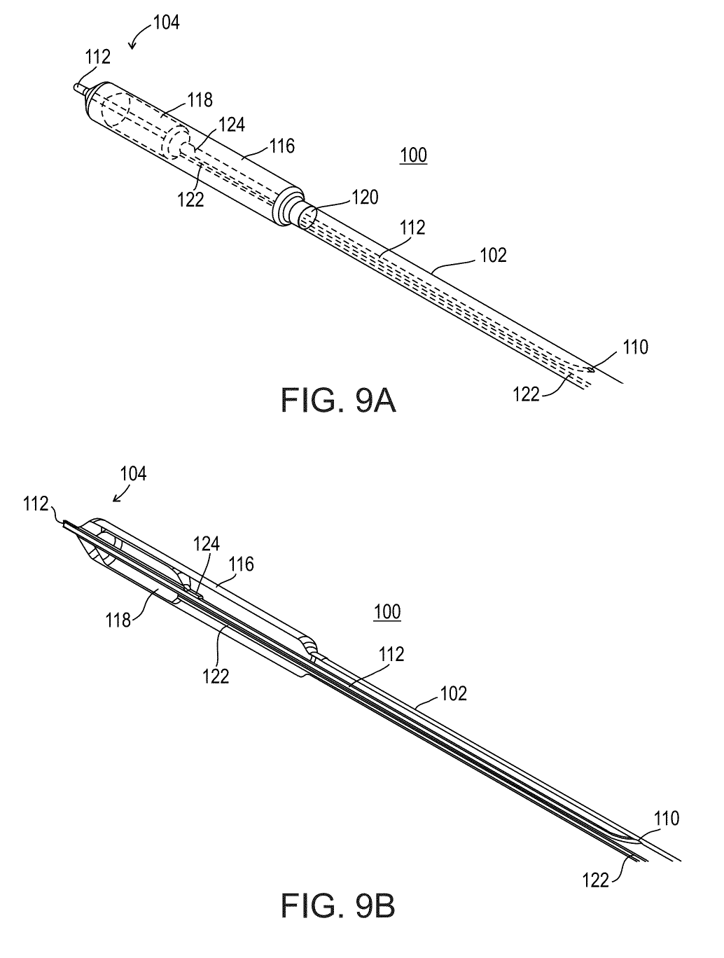

FIGS. 8a-9b illustrate configurations of the present system which include a short balloon conduit positioned side-by-side to, rather than concentric with, the guidewire conduit. These configurations of the present system are referred to herein as system 100.

FIGS. 8a-d illustrate an over-the-wire configuration of system 100, while FIGS. 9a-b illustrate a rapid exchange system (also referred to as monorail) configuration of system 100. Both systems are generally similar in size and function to systems 10 and 50 described above.

FIG. 8a illustrates a complete system 100 showing catheter 102 attached to balloons 116 and 118 (Balloon 116 shown in FIG. 8b) at distal end 104 and handle (port connector) 106 (shown in detail in FIG. 8d) at proximal end 108. It will be appreciated that the length of catheter body (between handle 106 and balloon 116 and 118) is not shown to scale and is substantially shortened for illustrative purposes.

Guidewire port 110 enters handle 106 from the side and connects to a conduit 112 which runs through catheter 102 and balloon 116 and 118 (FIG. 8b). Conduit 112 enables system 110 to be positioned over-the-wire. Handle 106 includes ports 114 and 115 for connecting handle 106 to inflation/deflation sources (syringe or pump).

Port 114 is in fluid communication with conduit 120 which is in turn in fluid communication with balloon 116 (shown in FIGS. 8c-d). Fluid pushed through port 114 can be used to inflate balloon 116, while fluid withdrawn through port 114 can be used to deflate balloon 116.

Port 115 is in fluid communication with conduit 122 which runs the length of handle 106 and parallel to conduit 112. Conduit 122 is in fluid communication with balloon 118 and thus can be used to inflate/deflate balloon 118 through port 115.

Conduit 122 is also used for moving balloon 118 within balloon 116 by pushing or pulling conduit 122 with respect to handle 106. To ensure that pushing/pulling of conduit 122 moves balloon 118 as a single unit, i.e. that balloon 118 does not shorten or lengthen under pulling or pushing forces, a tube 124 (FIGS. 8b-c) is attached (glued/welded) to balloon 118 and positioned (concentrically) over conduit 112. Tube 124 provides balloon 118 attached thereto with longitudinal rigidity and enables smooth movement of balloon 118 over conduit 112.

It will be appreciated that the locations of port 115 and 110 can be switched, positioning port 115 off the side of handle 106. However, such an arrangement is less preferred since requires that conduit 122 angle from port 115 to balloon 118. Since conduit also functions in moving balloon 118 within balloon 116, such an angle can increase friction and restrict movement.

FIGS. 9a-b illustrate a monorail (rapid exchange) configuration of system 100.

The arrangement of ports 114 and 115 in handle 106 and respective conduits 120 and 122 is similar to that shown in FIG. 8a. Port 110 is moved off handle 106 and onto catheter 102, thus enabling a rapid exchange set up of system 100. A rapid exchange configuration enables use of a shorter guidewire since the guidewire is threaded through an in port which is much closer to the effector end of system 100 (the balloons).

The present system can also be used to deliver a composition to the walls of the blood vessel. Such a composition can be, for example, a drug such as paclitaxel or sirolimus (rapamycin), or a bioadhesive.

Delivery of a composition from system 10 can be effected by coating balloon 16 with the composition or by including the composition in a reservoir formed in balloon 16 or preferably 18.

A coated configuration of system 10 can be fabricated using methods well known in the art, see, for example, Ruebben, [Interventional Cardiology, 2010; 5:74-6], Diehm [Tech Vasc Intery Radiol. 2010 March; 13(1):59-63], US 2011/0281019, US 2010/0324645, US 2011/0144578 and US 2010/0285085.

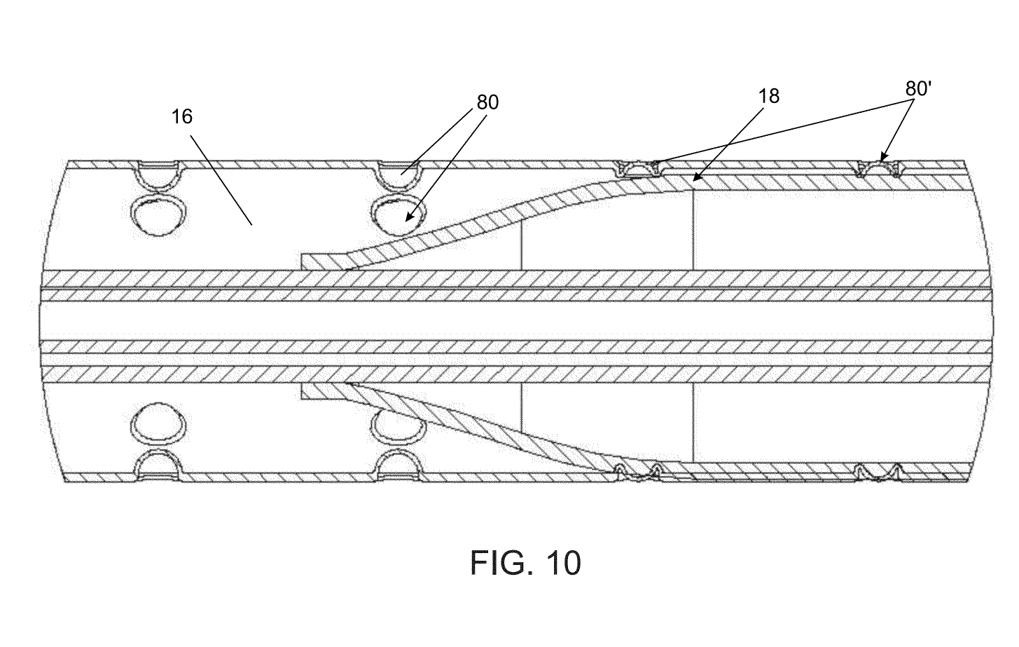

FIG. 10 illustrates one preferred configuration of a coated embodiment of system 10.

System 10 includes the balloon and catheter arrangement described above. External balloon 16 of system 10 of this embodiment of the present invention includes cavities (dimples/perforations) 80 in its external surface. Cavities 80 are configured for carrying a composition in dry, liquid or paste form. Approaches for preparing and loading a drug composition (e.g. paclitaxel) onto an angioplasty balloon are well known in the art (see references above). Each of cavities 80 can be loaded with 5-100 μl of a drug powder/solution and capped by a sugar, PLA or PGA cap.

Fabrication of cavities 80 can be effected by blow molding balloon 16 within an appropriate template or by cutting (e.g. via laser/blade) the external surface thereof following fabrication.

Balloon 16 can be folded (for delivery) such that cavities 80 are protected from exposure to the blood during delivery. This minimizes the amount of drug lost during delivery.

Once delivered to a target lesion, balloon 16 of system 10 is unfolded and inflated as described hereinabove.

The depth of cavities 80 decreases with an increase in inflation pressure of balloon 16. Above a specific inflation pressure (e.g. >15 atm), the wall of balloon 16 (which is semi compliant) stretches to flatten cavities 80 (depth equals zero) to expose the content of cavities 80. By controlling the inflation pressure of balloon 16, one can control the amount of drug released from each cavity 80. For example, initial inflation of balloon 16 (to 4-10 atm) can be used to release a first dose of the drug from each cavity 80, while additional inflation (over 10 atm) can be used to deliver a second dose (e.g. the remainder of the total dose carried by each cavity 80).

Alternatively, the drug can be delivered locally, i.e. from a subset of cavities 80′ by using internal balloon 18. Inflation of internal balloon 18 (as described hereinabove) can be used to extrude cavities 80′ (FIG. 8, right side) positioned at, for example, a site of residual stenosis to release a high local dose (optionally after release of an initial dose by inflation of balloon 16). Since balloon 18 inflates to higher pressure (e.g. 15-20 atm) and is smaller in volume (then balloon 16) it applies a larger force to a small area of the vessel (through a small area of balloon 16) and further enhances contact between cavities 80 and the vessel wall. Such inflation can facilitate complete and localized release of a drug from cavities 80 that are positioned over balloon 18.

Thus, in a drug delivery configuration, system 10 of the present invention provides several additional advantages including:

(i) cavities (capped or not) minimize drug loss during delivery;

(ii) controlled stepwise release of coated drug by controlling inflation pressure of external balloon; and

(iii) enhanced localized release of drug at sites of residual stenosis via internal balloon.

The present system can be packed for delivery using any approach known in the art. In order to minimize the external diameter of the catheter system and in particular of the balloon portion thereof and thus enable delivery of the catheter system into, for example, blood vessels having a diameter of less than 3 mm or blood vessels that narrow along their length (conical), the balloon portion of the present system is folded and packed in the proximal end of the external balloon and both are folded to a minimal final diameter. Such a packing configuration will result in a 3-4 Fr distal tip which is capable of going through a lesion. Once the external balloon is inflated it will further open the lesion and enable delivery of the internal balloon (by moving it within the external balloon) into the lesion.

Although the foregoing describes use of the present system in opening blocked blood vessels, it will be appreciated that the present system can also be used to open or widen other types of biological vessels including, for example, the urethra or ureters.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above descriptions, illustrate the invention in a non limiting fashion.

### Bench Testing of an Over-the-Wire Prototype

A prototype catheter system similar in configuration to that described with respect to FIGS. 8a-b was constructed and tested under bench conditions.

The prototype system was constructed as follows:

(i) a 0.024″×0.18″ nylon tube serving as a wire lumen was glued to the distal end of a 6 mm×100 mm nylon balloon serving as the external balloon;

(ii) a 20 mm length 0.034″×0.030″ polyimide tube serving as slider was internally glued to the distal and proximal ends of a 6 mm×20 mm nylon balloon serving as inner balloon;

(iii) a 0.020″×0.017″ stainless hypotube serving as inner balloon supply line and pusher was glued to the inner balloon proximal end tangent to the slider;

(iv) the inner balloon slider was thread over the wire lumen and the inner balloon was inserted into the external balloon through its proximal end;

(v) a 0.079″×0.071″ nylon tube serving as outer lumen was threaded over both the wire lumen and the hypotube and glued to the proximal end of the external balloon.

(vi) the outer tube and the guidewire tube were cut to length (about 100 cm) and glued to an off the shelf Y connector (Outer lumen to Y connector distal side, wire lumen to Y connector side port) while the stainless steel hypotube was inserted through the main port to serve as inner balloon drive handle; and

(vii) a 0.014″ wire was threaded through the side port and into the wire lumen.

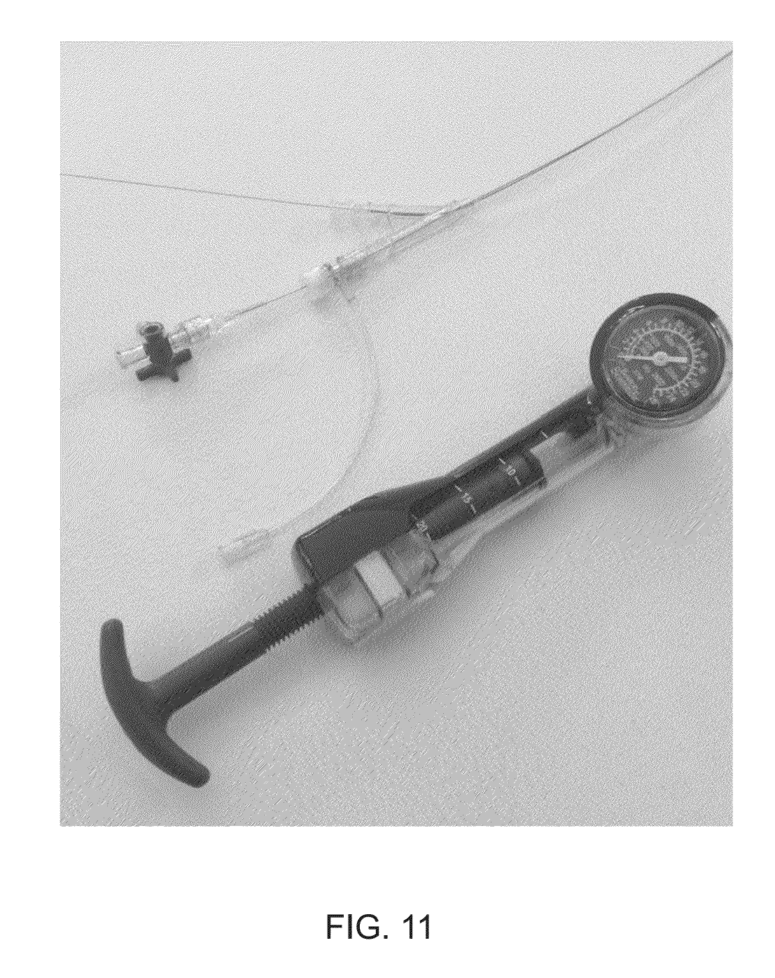

The catheter system was connected to the high pressure balloon pump (BSC Encore® 26 Inflation Device) shown in FIG. 11 and the external balloon were inflated to working pressure, following which internal balloon was moved within the external balloon by first pushing and then pulling the fluid conduit of the external balloon located and inflated to working pressure.

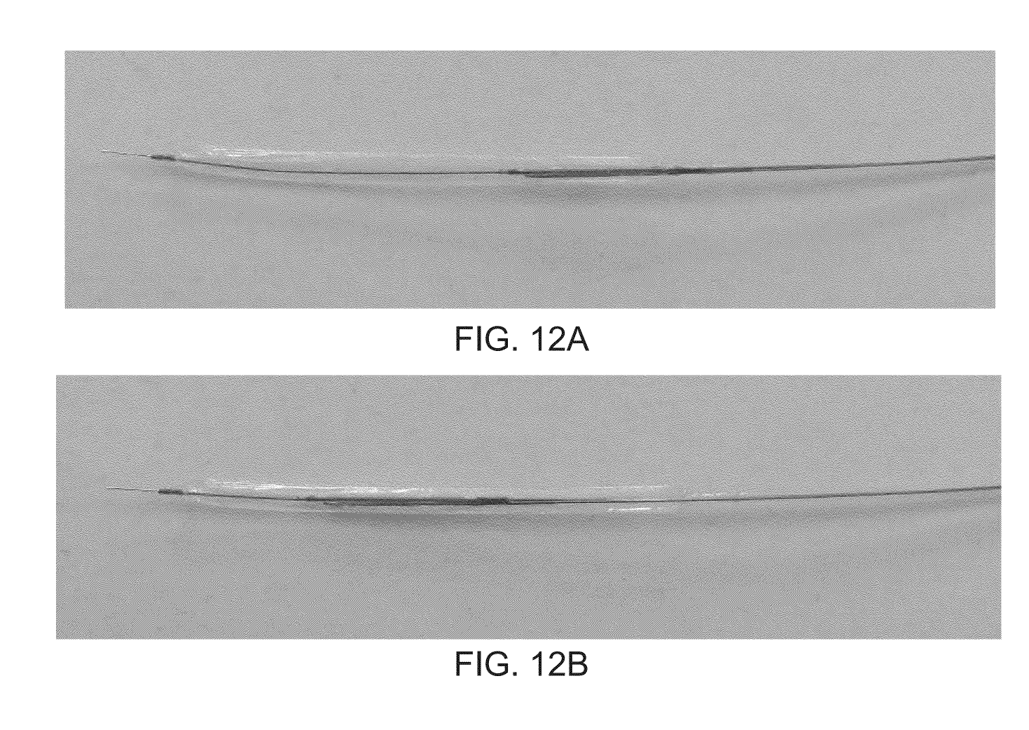

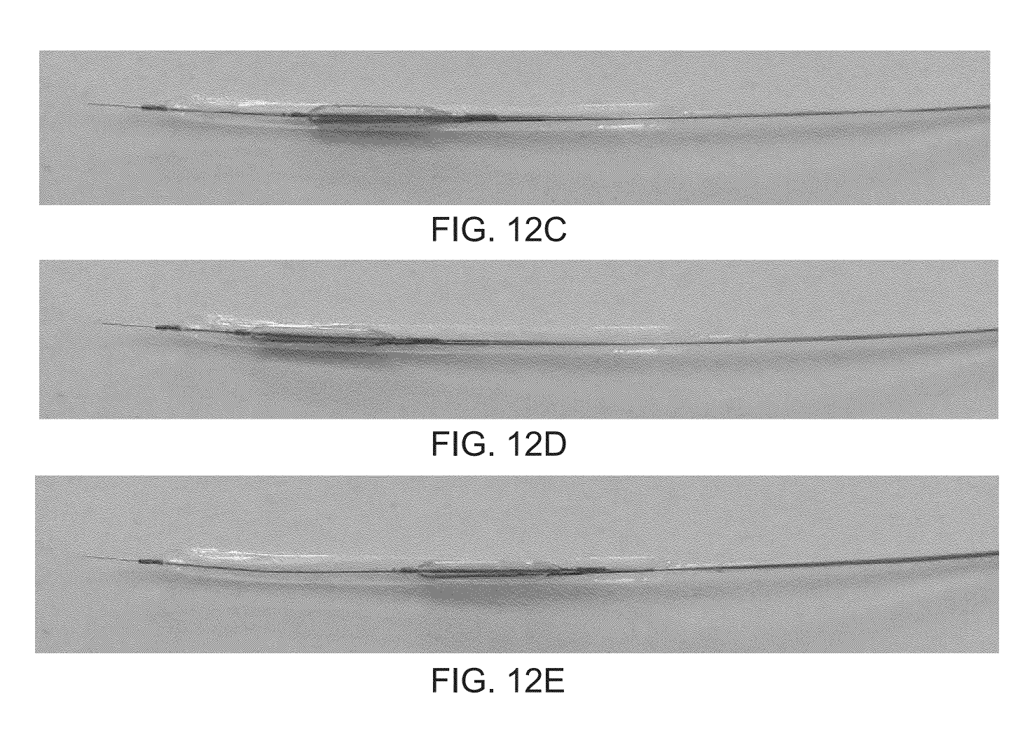

FIGS. 12a-e are a series of images showing: the system prior to inflation with both external and internal balloons being deflated and collapsed (FIG. 12a), external balloon inflation (FIG. 12b), internal balloon inflation (FIG. 12c) and movement of internal balloon with the external balloon being partially deflated (FIGS. 12d-e).

The external balloon was inflated to a pressure of 8 atm, while the internal balloon was inflated to a pressure of 12 atm. The internal balloon was slightly deflated and translated along the longitudinal axis of the external balloon. Both balloons were then deflated. Operation of the system through inflation, deflation and movement of internal balloon was deemed acceptable by an experienced user.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An angioplasty system comprising a catheter being mountable on a guide-wire, said catheter including a first balloon disposed around a second balloon, said second balloon being movable within said first balloon.

2. The system of claim 1, wherein said second balloon is movable along a longitudinal axis of said first balloon.

3. The system of claim 1, wherein said first balloon is a non- or semi-complaint balloon.

4. The system of claim 1, wherein said second balloon is a non-compliant balloon.

5. The system of claim 1, wherein said first balloon is inflatable to a pressure of up to about 12 atm.

6. The system of claim 1, wherein said second balloon is inflatable to a pressure of up to about 30 atm.

7. The system of claim 1, wherein a length of said second balloon is less than half than that of said first balloon.

8. The system of claim 1, wherein a conduit for inflating said first balloon is co-axial with a conduit for said guidewire.

9. The system of claim 1, wherein a conduit for inflating said second balloon is positioned side-by-side to a conduit of said guide-wire.

10. The system of claim 1, wherein said second balloon is movable within said first balloon by pulling or pushing said conduit with respect to said catheter.

11. The system of claim 1, wherein said catheter includes 2 tubes, a first tube attached to said first balloon and a second tube attached to said second balloon.

12. The system of claim 11, wherein said first tube includes a first lumen for inflating said first balloon and said second tube includes a second lumen for inflating said second balloon.

13. The system of claim 1, wherein an external surface of said first balloon is coated with a drug.

14. The system of claim 1, wherein an external surface of said first balloon includes cavities.

15. The system of claim 14, wherein said cavities are filled with a drug.

16. The system of claim 1, wherein said second balloon is attached to a tube coaxial with a conduit of said guidewire.

17. The system of claim 16, wherein a length of said tube is about a length of said second balloon.

18. The system of claim 1, wherein said catheter includes a valve for automatically releasing fluid from said first balloon when a pressure thereof exceeds a predetermined value.

19. The system of claim 12, further comprising a connector for fluidly connecting said first tube and said second tube to a single inflation source, said connector including a selector for selectively connecting said first lumen or said second lumen to said single inflation source.

20. A method of opening a stenosed vessel comprising:
(a) using a guidewire to deliver a catheter including a folded first balloon disposed around a folded second balloon to a site of stenosis in the vessel;
(b) inflating said first balloon within said stenosis in the vessel to thereby at least partially open said stenosis;
(c) moving said second balloon within said first balloon while maintaining said first balloon in position to thereby position said second balloon at a desired location within said stenosis; and
(d) inflating said second balloon thereby further opening said stenosis.

21. The method of claim 20, wherein step (c) is effected while said first balloon is inflated.

22. The method of claim 20, wherein said first balloon is coated with a drug and (a) releases a first dose of said drug.

23. The method of claim 22, wherein (d) releases a second dose of said drug from said first balloon.

24. The method of claim 20, wherein said folded second balloon is positioned within a proximal portion of said folded first balloon during (a).

25. The method of claim 20, wherein a diameter of said vessel varies along a length of said stenosis and (c) is used to compensate for said diameter variation.
